(19) United States
(12) Patent Application Publication
(10) Pub. No.: US 2014/0178315 A1
(43) Pub. Date: Jun. 26, 2014

(54) Topical Compositions Comprising Ionic Fluids

(71) Applicant: Arch Chemicals, Inc., Atlanta, GA (US)

(72) Inventors: James Vincent Gruber, Washington, CT (US); Vito Cataldo, Somerset, NJ (US); Khat Kevin Lou, East Brunswick, NJ (US)

(73) Assignee: Arch Chemicals, Inc., Atlanta, GA (US)

(21) Appl. No.: 14/136,315

(22) Filed: Dec. 20, 2013

Related U.S. Application Data
(60) Provisional application No. 61/739,791, filed on Dec. 20, 2012.

(51) Int. Cl. A61K 8/44; A61Q 19/00

(52) U.S. Cl. CPC A61K 8/44; A61Q 19/00
USPC 424/59; 514/784; 514/772.1; 424/70.1; 514/773

(57) ABSTRACT

The present invention relates to anhydrous or essentially anhydrous compositions intended for topical application on keratinaceous surfaces, wherein the composition includes a) an ionic fluid and b) a thickener. The compositions are advantageous for allowing the ionic fluid to act as the continuous phase of a topical formulation.

### RELATED APPLICATIONS

This application claims filing benefit of U.S. Provisional Patent Application Ser. No. 61/739,791, filed on Dec. 20, 2012, and which is incorporated herein by reference in its entirety

### FIELD OF INVENTION

The present invention generally relates to a topical pharmaceutical composition containing ionic fluids and thickening agents.

### BACKGROUND

Ionic fluids are ionic compounds that possess a melting temperature below 100° C. At least one ion in an ionic fluid is organic, which prevents the formation of a stable crystal lattice. Properties such as the melting point, viscosity and solubility in a variety of solvents are determined by specific structure. The existence of ionic salts and more specifically ionic fluids has been well documented (Freemantle, “Intro to Ionic Liquids,” M., 2010). Their physical and chemical properties are attractive for various applications. Ionic fluids are known to be stable, have good solvent properties, low color and a low vapor pressure. The search for new and different ionic fluids has led to the development of three generations of ionic fluids: (1) The focus of the first generation was mainly on their unique intrinsic physical and chemical properties, such as density, viscosity, conductivity, solubility, and high thermal and chemical stability. 2) The second generation of ionic fluids improved some of these physical and chemical properties, allowing the formation of “task-specific ionic liquids” which can have application as lubricants, energetic materials, and as more environmentally friendly reaction solvents. 3) The third generation of ionic fluids involved active pharmaceutical ingredients (API), which were used to produce ionic fluids with biological activity.

While many of the present applications for ionic liquids are not personal care related, these compounds have been used in the personal care market for hair conditioning. However, new ionic liquids are needed for use in personal care formulations that will deposit on the hair and skin from a variety of solvents with improved delivery efficiency. Of particular interest for such applications are ionic fluids that are liquid at ambient temperature.

One class of ionic fluids that are liquid at room temperature include amphoteric molecules joined with anionic molecules. Amphoteric molecules contain both cationic (+) and anionic (−) charge on the same molecule. Examples of such amphoteric molecules include, but are not limited to, betaine (also known as trimethylglycine), and carnitine. Ionic salts containing betaine are well known in the art. For example, U.S. Pat. No. 3,002,886 discloses solid ionic salts including betaine and salicylic acid intended for oral use. This patent fails to disclose the possible topical use of such ionic salts nor does it disclose the possible application of ionic liquids comprised of these two molecules. However, the topical use of ionic salts made from betaine and salicylic acid have been disclosed elsewhere [Arch Personal Care Technical Datasheet, Genti-Fol™, 2006].

WO 2011/079001 suggests the use of trimethylglycine and urea to form eutectic liquids in the presence of antiperspirant and deodorant actives. The application suggests that carboxylic acids can be added to the formulations, but the compositions solidify at ambient temperature.

US 2006/0166856 has suggested the use of ionic fluids in topical applications. In particular, US 2006/0166856 discloses ionic fluids in which one portion of the fluid is a cationic fragrance molecule. The ionic fluids are present in 0.1-20 wt % of the total composition. DE 102005056155 discloses shampoos containing surfactants and ionic fluids. These formulations also comprise between 0.1-20 wt % of the ionic fluid. However, formulations of US 2006/0166856 and DE 102005056155 also contain water and are not anhydrous.

What has not been disclosed in the prior art are compositions that are anhydrous, or essentially anhydrous, containing ionic fluids for topical use on keratineacous surfaces that are anhydrous or essentially anhydrous (less than 5% water), and that are liquid at room temperature.

Thus, there is a need for ionic fluids for use in personal care formulations that will deposit active ingredients on the hair and skin with improved delivery efficiency. In particular, there is a need for topical compositions containing ionic fluids that are liquid at ambient temperature, wherein ionic liquid effectively forms the entire continuous phase of the topical formulation.

### SUMMARY OF THE INVENTION

The present invention, therefore, is directed to ionic fluids for use in personal care formulations that will deposit active ingredients on the hair and skin with improved delivery efficiency. In one aspect, the present invention relates to topical compositions containing ionic fluids that are liquid at ambient temperature, wherein the ionic fluid effectively forms the entire continuous phase of the topical formulation. Another aspect of the present invention relates to the creation of yield stress in the ionic fluid by dissolving or dispersing a thickener into the ionic fluid. Yield stress in ionic fluids will allow the fluids to effectively suspend other ingredients, such as oils, pigments, sunscreens, micas, powders, waxes, beads and the like. These and other aspects will become apparent upon reading the following detailed description of the present invention.

### DESCRIPTION OF THE INVENTION

The present invention, therefore, is directed to compositions containing ionic fluids that are liquid at ambient temperature, and when included with thickeners create yield stress in the ionic fluid. As used herein, the term “ionic fluid” refers to ionic liquids and ionic liquid composites. The ionic liquid can comprise an anionic ionic liquid component and a cationic ionic liquid component. When the ionic liquid is in its liquid form, these components may freely associate with one another. As used herein, the term “ionic liquid composite” refers to a mixture of a salt (which can be solid at room temperature) with a proton donor (which can be a liquid or a solid at room temperature). Upon mixing, these components form a liquid at about 100° C. or less, and the mixture behaves like an ionic liquid.

Accordingly, the present invention is directed to a composition including a compound having the following general formula:

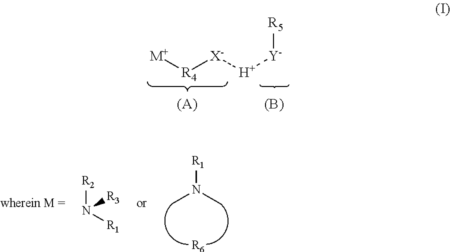

wherein R₁, R₂, R₃ and R₅ are each independently selected from alkyl group, substituted alkyl group, alkenyl group, substituted alkenyl group, alkynyl group, substituted alkynyl group, cycloalkyl group, substituted cycloalkyl group, cycloalkenyl group, substituted cycloalkenyl group, cycloalkynyl group, substituted cycloalkynyl group, aryl group, substituted aryl group, heterocyclic group and substituted heterocyclic group; R₄ is an alkylene group or substituted alkylene group; R₆ is selected from alkyl group, substituted alkyl group, alkenyl group, and substituted alkenyl group; and X and Y are each independently sulfonate, phosphate or carboxylate moieties.

The substituted alkyl group is an alkyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms; the substituted alkenyl group is an alkenyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms; the substituted alkynyl group is an alkynyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;

the substituted cycloalkyl group is a cycloalkyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxyl group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkenyl group is a cycloalkenyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkynyl group is a cycloalkynyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted aryl group is an aryl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxyl group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted heterocyclic group is a heterocyclic group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms; and
the substituted alkylene group is an alkylene group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms.

One embodiment of the present invention is the compound of formula (I) wherein R₁, R₂, R₃, and R₅ are each independently selected from alkyl group or substituted alkyl group, R₄ is a alkylene or substituted alkylene group, and X and Y are carboxylate moieties, wherein the substituted alkyl group and the substituted alkylene group are as defined above.

Exemplary compounds having structure (A), as shown in formula (I), include betaine, choline, carnitine, and the like. Exemplary compounds having structure (B), as shown in formula (I), include glycolic acid, malic acid, tartaric acid, kojic acid and the like.

More specifically, the present invention relates to a composition including a compound having the following formula:

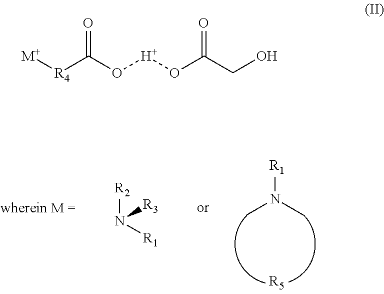

wherein R₁, R₂ and R₃ are each independently selected from alkyl group, substituted alkyl group, alkenyl group, substituted alkenyl group, alkynyl group, substituted alkynyl group, cycloalkyl group, substituted cycloalkyl group, cycloalkenyl group, substituted cycloalkenyl group, cycloalkynyl group, substituted cycloalkynyl group, aryl group, substituted aryl group, heterocyclic group and substituted heterocyclic group; and R₄ is an alkylene group or substituted alkylene group, and R₅ is selected from alkyl group, substituted alkyl group, alkenyl group, and substituted alkenyl group, wherein the substituted alkyl group, substituted alkenyl group, substituted alkynyl group, substituted cycloalkyl group, substituted cycloalkenyl group, substituted cycloalkynyl group, substituted aryl group, substituted heterocyclic group and the substituted alkylene group are as defined above.

Compounds having a structure (A) may be combined with compounds having a structure (B) in a desired ratio. For instance, the weight ratio of compounds having structure (A) to compounds having structure (B) may be from about 1/5 to less than about 5/1. For instance, the ratio may be from about 1/5, such as from about 1/4, such as from about 1/3, such as from about 1/2 to less than about 5/1, such as less than about 4/1, such as less than about 3/1, such as less than about 2/1.

Ionic fluid compositions in accordance with embodiments of the present invention can include thickening agents for creating yield stress in ionic fluids. While the ionic fluids work effectively by themselves as topical formulations, they are not typically able to suspend other desirable ingredients such as oils, powders, pigments, sunscreens, micas, waxes, beads, and the like, conventionally used in personal care formulations. For this reason, a composition in accordance with embodiments of the present invention includes at least one thickening agent dissolved or dispersed into the ionic fluid to create a rheological stress point, or yield stress, in the ionic fluid. Yield stress in an ionic fluid enables the fluid to suspend and support other cosmetically useful ingredients that would otherwise separate and settle out of the fluid. Yield stress can be measured using instruments such as rheometers (e.g., Bohlin Rheometer, Malvern Instruments, Worchestershire, UK).

Thickening agents used in ionic fluid compositions of the present invention may be present in an amount ranging from 0.01% by weight to about 9% by weight, such as in an amount greater than about 0.01% by weight, such as greater than about 0.1% by weight, such as greater than about 0.25% by weight, such as greater than about 0.5% by weight, such as greater than about 1% by weight and less than about 9% by weight, such as less than about 7% by weight, such as less than about 5% by weight, such as less than about 3% by weight, such as less than about 2% by weight, based on the weight of the total composition of the ionic fluid.

Exemplary thickening agents that can be dissolved or dispersed in ionic fluids compositions of the present invention include polymers, powders, esters, sugars, waxes, amides and the like. Exemplary polymers that can be used as thickening agents in compositions of the present invention include Carbopol Polymers (sold by Lubrizol Corporation, Wickliffe, Ohio) and Poloxamer Polymers (BASF), both of which are capable of dissolving completely into the ionic fluids and create yield stress. Carbopol Polymers are generally high molecular weight homopolymers and/or copolymers of an acrylic acid (e.g., a polyacrylic acid) crosslinked utilizing a crosslinking agent, such as a polyalkenyl polyether. Poloxamer Polymers are generally polyethylene oxide/polypropylene oxide block copolymers such as triblock copolymers comprising a central hydrophobic chain such as a polypropylene oxide chain flanked by two hydrophilic chains such as polyethylene oxide chains.

The present invention is also directed to a pharmaceutical composition comprising the compound of formula (I), a thickening agent, and at least one of a pharmaceutically acceptable carrier or a pharmaceutically acceptable adjuvant. In one embodiment, the present invention is directed to a topical composition comprising the compound of formula (I), a thickening agent, and, optionally, at least one therapeutic agent. Topical compositions in accordance with embodiments of the present invention may include ionic fluids of compound (I) in amounts of greater than about 0.01%, such as greater than about 1% by weight, such as greater than about 2% by weight, such as greater than about 4% by weight, such as greater than about 10% by weight, such as greater than about 25% by weight, such as greater than about 40% by weight and generally less than about 99% by weight, such as less than about 95% by weight, such as less than about 80% by weight, such as less than about 60% by weight, such as less than about 50% by weight, such as less than about 20% by weight, such as less than about 10% by weight, based on the weight of the overall topical composition. For instance, it should be understood that the amount of ionic fluid in the topical composition may vary depending on the specific application, for example, as a lotion, nail polish, lip gloss, hair treatment, cream, etc.

The present invention further provides a process for the preparation of ionic fluid compositions including a compound of formula (I), as defined above, and a thickening agent, which comprises the steps of blending equal or essentially equal molar weight percentages of powdered molecule (A) with powdered molecule (B), warming the blended powders to at least 30° C., 45° C. or 50° C., and allowing the ionic salt of formula (I) to form in situ. Dispersion or dissolution of the thickening agent can occur at the same time as the formation of the ionic fluid or after formation of the ionic fluid. Alternatively, thickening agents can be added after the ionic fluid has formed using mixing methods known to those skilled in the art. Additional optional ingredients may be added to the ionic fluid composition before or after the addition of the thickening agents to the ionic fluid.

Embodiments of the present invention relate to compositions that are anhydrous and that are liquid at room temperature. As used herein, the term “essentially anhydrous” means a composition containing less than 5% by weight of water, such as less than 1% by weight of water, such as less than 0.5% by weight water, such as less than 0.1% by weight, such as 0% by weight. The compositions of the present invention provide an anhydrous foundation capable of suspending or dissolving cosmetically active ingredients, and are useful for delivering these ingredients to keratineacous surfaces. Compositions in accordance with embodiments of the present invention comprising compounds of formula (I) and thickening agents are also capable of dissolving non-liquid ionic salts, such as a salt prepared from trimethylglycine and salicylic acid. This allows these actives to be incorporated into anhydrous formulations.

Cosmetically active ingredients that can be included in the ionic fluid compositions of the present invention are well known in the art, and are discussed in PCT application WO 97/39733, the contents of which are incorporated herein by reference in its entirety. Cosmetically active ingredients that can be dissolved in compositions of the present invention include: Feel modifiers, such as silicone oils, perfluoroether polymers or polymers such as Polyox® resins (water soluble polyethylene oxide polymer) and the like; Pigments and skin appearance modifying ingredients, such as iron oxide or mica and the like; Liquid sunscreens, such as, octylmethoxycinnimate or oxybenzone and the like; Powdered sunscreens, such as TiO₂ and the like; Natural and or synthetic oils, such as Panalene oils, mineral oils, avocado oil, olive oil and the like; Humectants, such as glycerin, propylene glycol, capryl glycol and the like; Skin keratolytic agents, such as salicylic acid, lactic acid or retinoic acid (and its derivatives such as retinol and retinyl palmitate) and the like; Moisturizers, such as petroleum jelly (Vaseline) or Propolis and the like; Antioxidants, such as EGCG (Epigallocatechin gallate), Resveratrol, Vitamin E, Pomiferin and the like; Extracellular matrix promoting ingredients, such as ascorbic acid, niacinamide and the like; Skin tone modifiers, such as kojic acid, hydroquinone, forskolin and the like; Surface active ingredients, such as octylglucoside, cocamidopropylbetaine, lecithin and the like; Microbial and infection control agents, such as ethanol, triclosan, zinc pyrithione, cetyl trimethylammonium chloride and the like; Anti-inflammatories, such as hydrocortisone, acetyl salicylic acid and the like; Odor control ingredients, such as aluminum salts, or Polyhexanide and the like; Hair and scalp modifying ingredients, such as Minoxidil (6-Piperidin-1-ylpyrimidine-2,4-diamine 3-oxide) and the like; Sebum influencing agents, such as Elubiol and the like; Esters, such as capric/capryl triglyceride, and the like; Waxes, such as bees wax, cholesterol, jojoba wax and the like; Proteins and peptides, such as Hexapeptide-11, Superoxide dismutase and the like; and Fragrances, such as blue cypress oil, citrulline and the like.

Ionic fluids can be used as a continuous phase of cosmetic emulsions and allow water insoluble cosmetic ingredients, such as betaine salicylate to be incorporated into cosmetics.

### EXAMPLES

A more complete understanding of the present invention can be obtained by referring to the following illustrative examples of the practice of the invention, which examples are not intended, however, to limit the invention.

### Example 1

### Synthesis of a Betaine/Glycolate Ionic Fluid

To a clean glass mortar, anhydrous betaine and anhydrous glycolic acid at a 1:2 molar ratio were added. The mixture was ground with a glass pestle for 1 minute to form a rough mixture and then heated in an oven to 105° C. for 2 hours. The solid mixture formed a clear fluid after 2 hours, which was mixed to form a homogeneous mixture and then allowed to cool to room temperature. The mixture remained a viscous fluid at room temperature and is a betaine glycolate ionic fluid.

### Example 2

### Synthesis of a Carnitine/Glycolate Ionic Fluid

Using the same process described in Example 1, an anhydrous ionic fluid composition comprising a 1:2 molar ratio of carnitine and glycolic acid was prepared. The mixture remained a viscous fluid at room temperature and is a carnitine glycolate ionic fluid.

### Example 3

### Synthesis of a Betaine/Malate Ionic Fluid

To a clean round bottom flask, anhydrous betaine and anhydrous malic acid at a 1:1 molar ratio were added. Sufficient isopropanol was then added and the mixture was heated to 85° C. until all solids were completely soluble and dissolved. The solution was allowed to stand and cool to room temperature. Once cooled, the mixture separated into two distinct layers—one isopropanol and the other the ionic fluid. The fluids were separated, and the ionic fluid was dried to remove excess isopropanol, and remained a viscous fluid at room temperature.

### Example 4

### Synthesis of a Carnitine/Malate Ionic Fluid

Using the same process described in Example 3, an anhydrous ionic fluid composition comprising a 1:1 molar ratio l-carnitine and malic acid was prepared. The mixture formed a viscous fluid at room temperature and is a carnitine malate ionic fluid.

### Example 5

### Solubility of Betaine Salicylate in Betaine Glycolate Ionic Fluid

This example provides a process for dissolving an ionic salt of betaine and salicylic acid in an anhydrous ionic fluid composition comprising betaine and glycolic acid. Using the same process described in Example 1, an ionic fluid of betaine glycolate was prepared. 1 g of betaine salicylate was added to 9 g of the ionic fluid betaine glycolate, and the solution was mixed at room temperature for 1 minute to disperse the betaine salicylate. The resulting mixture was heated in an oven to 105° C. for 2 hours. The mixture formed a clear fluid after 2 hours, which was mixed to form a homogeneous liquid and then allowed to cool to room temperature. The mixture remained a viscous fluid at room temperature.

### Example 6

### Solubility of Betaine Kojiate in Betaine Glycolate Ionic Fluid

Using the same process described in Example 5, an ionic salt comprising betaine and kojic acid was dissolved in an anhydrous ionic fluid composition comprising betaine and glycolic acid. 3 g of the ionic salt betaine kojiate was added to 21 g of the ionic fluid betaine glycolate prepared using the process described in Example 1, and mixed at room temperature for 1 minute to disperse the betaine salicylate. The mixture was heated in an oven to 105° C. for 2 hours. The mixture formed a clear fluid after 2 hours, which was mixed until a homogeneous mixture was formed and then allowed to cool to room temperature. The mixture remained a viscous fluid at room temperature.

### Example 7

### Betaine/Glycolate Ionic Fluid Thickened with Carbopol Polymer

The anhydrous ionic fluid of Example 1 was thickened by dissolving a crosslinked polyacrylic acid polymer in the following proportions:

| Ingredient | Weight % | Function |
|---|---|---|
| Ionic Fluid of Example 1 | 95.00 | |
| Crosslinked polyacrylic acid | 5.00 | Thickening agent |
| (Carbopol ETD 2050) | | |
| Total | 100.00 | |

The thickening agent was added slowly into the ionic fluid while stirring, and the resulting solution was mixed until the gelling agent dissolved in the ionic fluid to form a homogenous liquid.

Using the same process described above, anhydrous ionic fluid of Example 1 was also thickened by using the following thickening polymers: Rapithix® A-60 (INCI—Sodium Polyacrylate (and) Hydrogenated Polydecene (and) Trideceth-6); Simugel EG (INCI—Sodium acrylate/acryloyldimethyl taurate copolymer & Isohexadecane & Polysorbate 80); and Sepigel 305 (INCI—Polyacrylamide and C13-14 isoparaffin and laureth-7).

### Example 8

### Cosmetic Formulations

Cosmetic formulations in the form of a lotion, cream, undereye treatment, lipstick, silicone-containing gel, ionic fluid-in-silicone emulsion, silicone-in-Ionic fluid emulsion, Fomblin-in-ionic fluid emulsion, anhydrous barsoap, toothpaste, nail treatment/polish, and an anhydrous hair treatment were made using the thickened anhydrous ionic fluid composition of Examples 1 and 2 and components shown below for each cosmetic formulation. Each of the following formulations was further blended with Silicone fluid, Vegetable Oil, synthetic esters, Argon Oil, Hydrocarbon, petrolatum, natural gums, natural waxes, synthetic waxes, natural sugar esters, natural emulsifiers, and/or synthetic emulsifiers.

### Lotion:

| Ingredient | % | Function |
|---|---|---|
| Betaine Glycolate | 76.00 | |
| Cetearyl Alcohol & Cetearth-20 | 2.50 | Emulsifier |
| Polyglyceryl-10 Stearate | 1.50 | Emulsifier |
| Ethylhexyl Palmiate | 20.00 | Emollient |
| Total | 100.00 | |

The ionic fluid was heated to 65° C. while mixing slowly. Ethylhexyl Palmitate, Cetearyl Alcohol & Ceteareth-20, and Polyglyceryl-10 stearate were added to the heated ionic fluid, and mixed until a homogenous mixture was formed.

### Cream:

| Ingredient | % | Function |
|---|---|---|
| Phase (A) | | |
| Betaine Glycolate | 74.00 | |
| Phase (B) | | |
| Cetearyl Alcohol & Cetearth-20 | 2.50 | Emulsifier |
| Crosslinked polyacrylic acid (Carbopol ETD 2050) | 2.00 | Thickener |
| Polyglyceryl-10 Stearate | 1.50 | Emulsifier |
| Ethylhexyl Palmiate | 20.00 | Emollient |
| Total | 100.00 | |

The Phase (A) ionic fluid was heated slowly to 65° C. in a container. In a separate container, Phase (B) ingredients were combined and heated to 70° C. Phase (B) was then added slowly to phase (A) ionic fluid and mixed until a homogenous mixture was formed.

### Under Eye Treatment:

| Ingredient | % | Function |
|---|---|---|
| Betaine Glycolate | 75.00 | |
| Polyglycerol-10 Laurate | 15.00 | Emulsifier |
| Organic Argan Oil | 10.00 | Natural Oil |
| Total | 100.00 | |

Organic Argan Oil and Polyglycerol-10 Laurate were added slowly into the ionic fluid while stirring and mixed until a homogenous mixture was formed.

### Lipstick:

| Ingredient | % |
|---|---|
| Phase (A) | |
| Musol SD | 0.25 |
| Candelilla Wax | 15.00 |
| Liquiwax DIADD* | 13.00 |
| Ivarlan 3100 | 5.00 |
| Ivarlan 3001 | 2.50 |
| Lanolin Light USP | |
| Multiwax W-835 | 2.50 |
| Yellow Beeswax | 2.00 |
| Lipocol C | 1.00 |
| Vitamin E Acetate | 0.10 |
| Propylparaben | 0.10 |
| Methylparaben | 0.30 |
| Phase (B) | |
| Flavor | q.s. |
| Betaine Glycolate | 5.00 |
| Phase (C) | |
| Color Grind | |
| Crystal O | 49.45 |
| D&C Red #6 FHC | 5.70 |
| D&C Red #7 FHC | 0.60 |
| Titanium Dioxide | 2.50 |
| FHC | |
| Total | 100.00 |

Phase (A) ingredients were heated to 80° C. in suitable container and until a homogenous mixture was formed. Phase (B) ingredients were slowly added to the homogeneous mixture of Phase (A) ingredients and mixed until a uniform mixture was formed. Phase (C) ingredients were added slowly to the uniform mixture and mixed until a homogenous mixture was formed, which was then poured into a lipstick mold.

### Lip Gloss:

| Ingredient | % |
|---|---|
| Phase (A) | |
| Sunflower Oil | Q.S |
| Sweet Almond Oil | 17.80 |
| Cetearyl Alcohol | 5.00 |
| Jojoba Esters 30 | 12.00 |
| Beeswax | 5.00 |
| Carnauba Wax SP 63 | 3.50 |
| Jojoba Esters 60 | 6.00 |
| Tocopheryl Acetate | 0.10 |
| FD&C Yellow No. 6 Al. Lake | 0.10 |
| Phase (B) | |
| Betaine Glycolate | 5.00 |
| Total | 100.00 |

Phase (A) ingredients were heated to 80° C. while mixing until a homogenous mixture was formed. Phase (B) Betaine Glycolate was slowly added to the Phase (A) mixture and mixed until a homogenous mixture was formed. The resulting mixture was poured into a lip gloss container.

### Ionic Fluid-In-Silicone Emulsion:

| Ingredient | % | Function |
|---|---|---|
| Cetyl PEG/PPG-10/1 Dimethicone (Abil EM-90) | 8.71 | Emulsifier |
| Cyclopentasiloxane/Cyclohexasiloxane blend | 36.54 | Emollient |
| (DC 345) | | |
| Betaine Glycolate | 54.75 | |
| Total | 100.00 | |

Cetyl PEG/PPG-10/1 Dimethicone (Abil EM-90) and the Cyclopentasiloxane/Cyclohexasiloxane blend (Dow Corning 345) were placed in a suitable container and mixed with a Homogenizer. Ionic fluid of Example 1 was slowly added to the homogenous mixture of Cetyl PEG/PPG-10/1 Dimethicone (Abil EM-90) and the Cyclopentasiloxane/Cyclohexasiloxane blend (Dow Corning), and mixed until a homogenous mixture was obtained.

Using the same process described above, additional anhydrous ionic fluid-in-silicone emulsions were prepared using the following emulsifiers: SF 1528 (INCI—Cyclopentasiloxane (and) PEG/PPG-20/15 Dimethicone); SF 1328 (INCI—Cyclomethicone (and) PEG/PPG-20/15 Dimethicone); Abil WE-09 (INCI—Polyglyceryl-4 Isostearate; Cetyl PEG/PPG-10/1; Dimethicone; Hexyl Laurate); and KSG-240 (INCI—Cyclopentasiloxane and PEG 0/15 dimethicone crosspolymer).

### Silicone-In-Ionic Fluid Emulsion:

| Ingredient | % | Function |
|---|---|---|
| Phase (A) | | |
| Montanov 82 | 4.46 | Emulsifier |
| DC 345 | 22.26 | Emollient |
| DC 200 | 4.45 | Emollient |
| Phase (B) | | |
| Betaine Glycolate | 68.83 | |
| Total | 100.00 | |

Phase (A) ingredients were combined and heated to 65° C. Phase (B) Ionic Fluid was slowly added to the heated Phase (A) mixture and mixed until a homogenous mixture was formed.

### Fomblin-In-Ionic Fluid Gel:

| Ingredient | % |
|---|---|
| Phase (A) | |
| Fomblin | 5.00 |
| Betaine Glycolate | 94.75 |
| Phase (B) | |
| Crosslinked polyacrylic acid (Carbopol ETD 2050) | 0.25 |
| Total | 100.00 |

Phase (A) ingredients were combined and mixed until a uniform mixture was formed. Crosslinked polyacrylic acid (Carbopol ETD 2050) was slowly added to the uniform mixture of Phase (A) ingredients and mixed until a homogenous mixture was formed.

### Anhydrous Bar Soap:

| Ingredient | % |
|---|---|
| ZETESAP ST 5251* | 95 |
| (natural soap base) | |
| Phase (B) | |
| Betaine Glycolate | 5.00 |
| Total | 100.00 |

*Disodium Lauryl Sulfosuccinate(and)Sodium Coco-Sulfate(and) Triticum Vulgare (Wheat) Starch (and) Cetearyl Alcohol (and) Paraffin (and) Cocamidopropyl Betaine (and) Sodium Laureth Sulfate (and) Glyceryl Stearate (and) Titanium Dioxide

Phase (A) Soap base was placed in suitable container and heated to 75-80° C. Phase (B) ionic Fluid was added slowly to the Phase (A) soap base and mixed until homogenous mixture was formed. The resulting mixture was then molded into a desired shape.

### Toothpaste:

| Ingredient | % |
|---|---|
| Glycerin | 1.81 |
| Eugenol | 0.15 |
| Peppermint | 0.15 |
| Spearmint | 0.25 |
| Cornstarch | 12.00 |
| Sodium Bicarbonate | 35.00 |
| Sodium Furoride | 0.15 |
| Betaine Glycolate | 5.00 |
| Polyglycerol ester (Polyaldo 10-1-CC) | 0.75 |
| Hydrogenated Soybean Oil | Q.s |
| Total | 100.00 |

Flavoring oils and oil extracts were combined with glycerin and mixed until a homogenous mixture was formed. Corn Starch was added to the mixture and mixed until a thickened homogenous mixture was formed. The thickened mixture was kneaded to form a dough-like mass and allowed to dry at about 115-125 F for about 8-12 hours.

Dried materials were ground into powder and combined with sodium fluoride and Polyalso 10-1-CC (Polyglycerol ester). The powder mixture was then whipped together with Hydrogenated Soybean Oil and Betaine Glycolate until a cream having the desired consistency was obtained.

### Nail Treatment/Polish:

| Ingredient | % |
|---|---|
| Suspending Lacquer SLF-2* | Q.s |
| Biju Ultra UXD** | 2.00 |
| Flamenco Ultra Sparkle 4500 | 1.00 |
| Red 6 lake & Red 7 lake & Red 34 lake | 8.75 |
| In Nitrocellulose base | |
| Thinner | 3.25 |
| Betaine Glycolate | 5.00 |
| Total | 100.00 |

*(Butyl Acetate (and) Toluene (and) Nitrocellulose (and) Tosylamide/Formaldehyde Resin (and) Isopropyl Alcohol (and) Dibutyl Phthalate (and) Ethyl Acetate (and) Camphor (and) n-Butyl Alcohol (and) Silica (and) Quaternium-18 Hectorite)
**Butyl Acetate (and) Bismuth Oxychloride (and) Nitrocellulose (and) Toluene (and) Isopropyl Alcohol (and) Stearalkonium Hectorite.

### Anhydrous Hair Treatment:

| Ingredient | % |
|---|---|
| Mineral Oil | Q.S |
| Mineral Oil (and) Hydrogenated Styrene Isoprene/ | 40.00 |
| Butadiene Copolymer | |
| Betaine Glycolate | 5.00 |
| Mica (and) Titanium Dioxide | 0.10 |
| Pluronic L92 (Poloxamer 282) | 5.00 |
| Total | 100.00 |

The ingredients were added to a contained in the order listed above and slowly mixed between each addition.

As such, those skilled in the art will appreciate that the conception upon which this disclosure is based may readily be utilized as a basis for the designing of other structures, methods and systems for carrying out the several purposes of the present invention. It is important, therefore, that equivalent constructions insofar as they do not depart from the spirit and scope of the present invention, are included in the present invention.

We claim:

1. A topical composition for application to keratinaceous surfaces comprising:
an ionic fluid of formula (I), 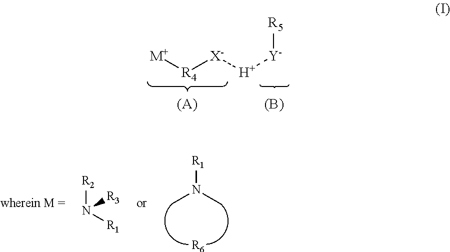
wherein R₁, R₂, R₃ and R₅ are each independently selected from alkyl group, substituted alkyl group, alkenyl group, substituted alkenyl group, alkynyl group, substituted alkynyl group, cycloalkyl group, substituted cycloalkyl group, cycloalkenyl group, substituted cycloalkenyl group, cycloalkynyl group, substituted cycloalkynyl group, aryl group, substituted aryl group, heterocyclic group and substituted heterocyclic group,
R₄ is an alkylene group or substituted alkylene group,
R₆ is selected from alkyl group, substituted alkyl group, alkenyl group, and substituted alkenyl group, and
X and Y are each independently sulfonate, phosphate or carboxylate moieties; and
a thickening agent.

2. The composition of claim 1, wherein:
the substituted alkyl group is an alkyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted alkenyl group is an alkenyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted alkynyl group is an alkynyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkyl group is a cycloalkyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxyl group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkenyl group is a cycloalkenyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkynyl group is a cycloalkynyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted aryl group is an aryl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxyl group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted heterocyclic group is a heterocyclic group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms; and
the substituted alkylene group is an alkylene group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms.

3. The composition of claim 1, wherein R₁, R₂, R₃, and R₅ are each independently selected from alkyl group and substituted alkyl group, and X and Y are each independently sulfonate, phosphate or carboxylate moieties.

4. The composition of claim 1, wherein the compound of formula (I) is betaine-glycolate.

5. The composition of claim 1, wherein the compound of formula (I) is betaine-lactate.

6. The composition of claim 1, wherein the compound of formula (I) is betaine-malate.

7. The composition of claim 1, wherein the compound of formula (I) is betaine-tartarate.

8. The composition of claim 1, wherein the compound of formula (I) is caritine glycolate

9. The composition of claim 1, wherein the thickening agent is present in an amount between about 0.01% to 9% by weight of the total composition.

10. The composition of claim 1, wherein the thickening agent is present in an amount of between 0.1% and 5% by weight of the total composition.

11. The composition of claim 1, wherein the thickening agent is present in an amount of between 0.5% and 2% by weight of the total composition.

12. The composition of claim 1, wherein the thickening agent is selected from a group consisting of polymers, powders, esters, sugars, waxes, and amides.

13. The composition of claim 1, wherein the thickening agent is selected from a group consisting of a polyacrylic acid and a polyethylene oxide/polypropylene block copolymer.

14. The composition of claim 1, further comprising at least one of feel modifiers, pigments and skin appearance modifying agents, liquid sunscreens, powdered sunscreens, oils, humectants, skin keratolytic agents, moisturizers, antioxidants, extracellular matrix promoting agents, skin tone modifiers, surface active agents, microbial and infection control agents, antiinflammatories, odor control agents, hair and scalp modifying agents, esters, waxes, proteins and peptides, and fragrances.

15. The composition of claim 1, further comprising an amount of water from about 0% to about 5% by weight relative to the total weight of the composition.

16. An anhydrous composition for application to keratinaceous surfaces comprising:
an ionic fluid of formula (I), 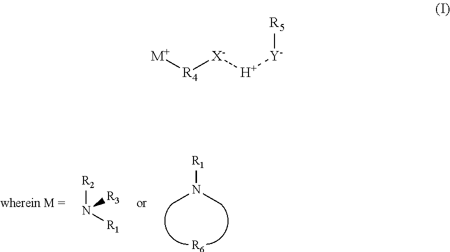
wherein R₁, R₂, R₃ and R₅ are each independently selected from alkyl group, substituted alkyl group, alkenyl group, substituted alkenyl group, alkynyl group, substituted alkynyl group, cycloalkyl group, substituted cycloalkyl group, cycloalkenyl group, substituted cycloalkenyl group, cycloalkynyl group, substituted cycloalkynyl group, aryl group, substituted aryl group, heterocyclic group and substituted heterocyclic group,
R₄ is an alkylene group or substituted alkylene group,
R₆ is selected from alkyl group, substituted alkyl group, alkenyl group, and substituted alkenyl group, and
X and Y are each independently sulfonate, phosphate or carboxylate moieties;
a thickening agent; and
water in an amount from about 0% to about 5% by weight relative to the total weight of the composition.

17. The anhydrous composition of claim 16, wherein:
the substituted alkyl group is an alkyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted alkenyl group is an alkenyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted alkynyl group is an alkynyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkyl group is a cycloalkyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxyl group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkenyl group is a cycloalkenyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkynyl group is a cycloalkynyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted aryl group is an aryl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxyl group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted heterocyclic group is a heterocyclic group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms; and
the substituted alkylene group is an alkylene group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms.

18. The anhydrous composition of claim 16, wherein R₁, R₂, R₃, and R₅ are each independently selected from alkyl group and substituted alkyl group, and X and Y are each independently sulfonate, phosphate or carboxylate moieties.

19. A method for preparing the composition of claim 1, said method comprising the steps of:
blending a first compound of formula (II) with a second compound having a formula R₅—YH, 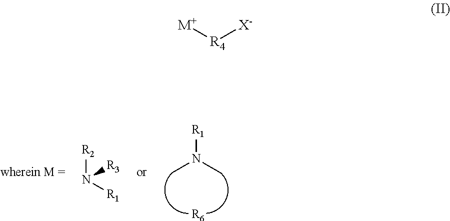
wherein R₁, R₂, R₃ and R₅ are each independently selected from alkyl group, substituted alkyl group, alkenyl group, substituted alkenyl group, alkynyl group, substituted alkynyl group, cycloalkyl group, substituted cycloalkyl group, cycloalkenyl group, substituted cycloalkenyl group, cycloalkynyl group, substituted cycloalkynyl group, aryl group, substituted aryl group, heterocyclic group and substituted heterocyclic group, R₄ is an alkylene group or substituted alkylene group, R₆ is selected from alkyl group, substituted alkyl group, alkenyl group, and substituted alkenyl group, and X and Y are each independently sulfonate, phosphate or carboxylate moieties,
wherein the first and second compound form a blended mixture; and
heating the blended mixture to a predetermined temperature to form the compound of formula (I); and
mixing a thickening agent with the first and second compounds.

20. The method of claim 19, wherein the thickening agent is mixed with the first and second compounds after the blending step.

21. The method of claim 19, wherein the thickening agent is mixed with the first and second compounds during the blending step.

22. The method of claim 19, wherein the blended mixture is heated to a temperature of about 30° C.

23. The method of claim 19, wherein the blended mixture is heated to a temperature of about 45° C.

24. The method of claim 19, wherein the blended mixture is heated to a temperature of about 50° C.

25. A topical composition for application to keratinaceous surfaces comprising:
an ionic fluid of formula (III), 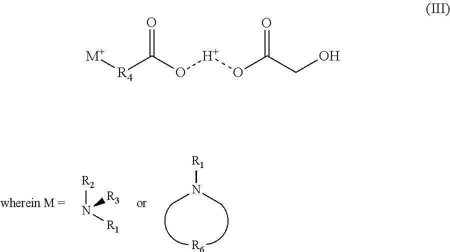
wherein R₁, R₂ and R₃ are each independently selected from alkyl group, substituted alkyl group, alkenyl group, substituted alkenyl group, alkynyl group, substituted alkynyl group, cycloalkyl group, substituted cycloalkyl group, cycloalkenyl group, substituted cycloalkenyl group, cycloalkynyl group, substituted cycloalkynyl group, aryl group, substituted aryl group, heterocyclic group and substituted heterocyclic group, R₄ is an alkylene group or substituted alkylene group, and R₅ is selected from alkyl group, substituted alkyl group, alkenyl group, and substituted alkenyl group; and
a thickening agent.

26. The composition of claim 25, wherein:
the substituted alkyl group is an alkyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted alkenyl group is an alkenyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted alkynyl group is an alkynyl group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkyl group is a cycloalkyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxyl group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkenyl group is a cycloalkenyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted cycloalkynyl group is a cycloalkynyl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms;
the substituted aryl group is an aryl group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxyl group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms; and
the substituted heterocyclic group is a heterocyclic group substituted with at least one radical independently selected from the group consisting of alkyl groups, alkenyl groups, alkynyl groups, cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms; and
the substituted alkylene group is an alkylene group substituted with at least one radical independently selected from the group consisting of cycloalkyl groups, cycloalkenyl groups, cycloalkynyl groups, aryl groups, heterocyclic groups, hydroxy group, alkoxy groups, alkenyloxy groups, alkynyloxy groups, cycloalkoxy groups, cycloalkenyoloxy groups, cycloalkynyloxy groups, aryloxy groups, alkylcarbonyloxy groups, cycloalkylcarbonyloxy groups, cycloalkenylcarbonyloxy groups, cycloalkynylcarbonyloxy groups, arylcarbonyloxy groups, thiol group, alkylthio groups, cycloalkylthio groups, cycloalkenylthio groups, cycloalkynylthio groups, arylthio groups, formyl group, acyl groups, carbamoyl groups, amino group, amino groups substituted with at least one alkyl group, alkenyl group or alkynyl group, acylamino groups, N-acyl-N-alkyl amino groups, N-acyl-N-alkenyl amino groups, N-acyl-N-alkynyl amino groups, N-acyl-N-cycloalkyl amino groups, N-acyl-N-cycloalkenyl amino groups, N-acyl-N-aryl amino groups, nitro group, heterocyclic groups and halogen atoms.
